SEQUENCE LISTING (9 sequences)

SEQ ID NO: 1 - DNA, 23 bp, synthetic construct
  misc_feature 1..23: note = primer
  source 1..23: mol_type = other DNA
ggagctagaacgattcgcagtta

SEQ ID NO: 2 - DNA, 25 bp, synthetic construct
  misc_feature 1..25: note = primer
  source 1..25: mol_type = other DNA
ggttgtagctgtcccagtatttgtc

SEQ ID NO: 3 - DNA, 30 bp, synthetic construct
  misc_feature 1..30: note = probe
  source 1..30: mol_type = other DNA
acagccttctgatgtttctaacaggccagg

SEQ ID NO: 4 - DNA, 22 bp, synthetic construct
  misc_feature 1..22: note = primer
  source 1..22: mol_type = other DNA
tctccattccctatgttcatgc

SEQ ID NO: 5 - DNA, 22 bp, synthetic construct
  misc_feature 1..22: note = primer
  source 1..22: mol_type = other DNA
gttcccgcagaatggtgaggtg

SEQ ID NO: 6 - DNA, 20 bp, synthetic construct
  misc_feature 1..20: note = probe
  source 1..20: mol_type = other DNA
atgttcctcggaccaacttg

SEQ ID NO: 7 - DNA, 20 bp, synthetic construct
  misc_feature 1..20: note = primer
  source 1..20: mol_type = other DNA
agggaactgtgggatgactg

SEQ ID NO: 8 - DNA, 20 bp, synthetic construct
  misc_feature 1..20: note = primer
  source 1..20: mol_type = other DNA
gaacacctgagcctttgagc

SEQ ID NO: 9 - DNA, 20 bp, synthetic construct
  misc_feature 1..20: note = probe
  source 1..20: mol_type = other DNA
gaacacctgagcctttgagc

(19) United States
(12) Patent Application Publication
(10) Pub. No.: US 2025/0027042 A1
(43) Pub. Date: Jan. 23, 2025

(54) Rapid T-Cell Manufacturing

(71) Applicant: Kure.AI, Inc., Shaker Heights, OH (US)

(72) Inventor: David Wald, Shaker Heights, OH (US)

(21) Appl. No.: 18/714,302

(22) PCT Filed: Dec. 22, 2022
(86) PCT No.: PCT/US2022/053815

§ 371 Date: May 29, 2024

Related U.S. Application Data
(60) Provisional application No. 63/292,843, filed on Dec. 22, 2021.

(51) Int. Cl. C12N 5/0783; C12N 5/00; C12N 15/86

(52) U.S. Cl. CPC C12N 5/0636; C12N 5/0018; C12N 15/86; C12N 2501/2307; C12N 2501/2315; C12N 2501/51; C12N 2501/515; C12N 2740/10043; C12N 2740/15043; C12N 2750/14143

(57) ABSTRACT

The present disclosure provides methods for rapid manufacture of a genetically modified immune effector cell such as a T-cell. The methods allow rapid (<1day) production of genetically modified immune effector cells such as CAR T-cells.

### RELATED APPLICATIONS

This is a utility patent application claiming the benefit of priority in U.S. Provisional Application Ser. No. 63/292,843 filed Dec. 22, 2021, the entire disclosure of which is incorporated herein by reference.

### REFERENCE TO SEQUENCE LISTING SUBMITTED ELECTRONICALLY

A sequence listing electronically submitted with the present application as an XML file named 2459S_005WO.xml, created on 12-22-2022 and having a size of 13000 bytes, is incorporated herein by reference in its entirety.

### TECHNICAL FIELD

The disclosure relates to a rapid T cell manufacturing workflow that enables the production of genetically manipulated T cell products in less than 1 day using viral mediated transduction and also provides innovation in product release testing to facilitate roadblocks in utilization of this product clinically.

### BACKGROUND

T cell therapy has shown enormous potential in the treatment of diseases, particularly cancer, infectious diseases and autoimmune diseases. One method to enhance T cell therapy is to genetically modify T cells using viral mediated gene transfer to enhance their activity and/or specificity to the desired target cells. For example, the expression of chimeric antigen receptors (CARs) on T cells using lentivirus and retrovirus has shown enormous potential in cancer therapy. Autologous T cells that express a chimeric antigen receptor (CAR-T cells) particularly directed against CD19 have exhibited significant efficacy in patients with relapsed or refractory B cell malignancies.

The potential of genetically engineered T cells is highlighted by the remarkable clinical success of autologous CD19 CAR-T cells in relapsed/refractory Non-Hodgkin lymphomas (NHL) and acute lymphoid leukemia (ALL). Aggressive, relapsed, or refractory disease is treated with high dose therapy followed by autologous stem cell rescue if disease is still chemotherapy sensitive (1, 2). Unfortunately, up to 50% of patients relapse or become refractory and the outcomes of the relapsed/refractory patients are poor with traditional chemotherapy (ex. 7% complete response rate (CR) and 6.3 month median overall survival (OS) (3)). CD19 CAR-T therapies in contrast have demonstrated significantly improved outcomes (ex. >50% CR and >50% OS at 1 year (4)) in these patients.

Despite the FDA approval of commercial CD19 and BCMA CAR-T cell products in the United States as well as regulatory approval in many other countries worldwide, the existing therapies remain cost prohibitive for use in the majority of the world and pose a significant economic burden even in the US. The current cost for manufacturing autologous CAR-T products at pharmaceutical companies has been estimated at ˜100 k USD per patient (5). In addition to cost, the manufacturing process is slow leading to undesirable delays in patient treatment. The current FDA approved products take ˜3-6 weeks or longer to be delivered to the patient that leads to disease progression in a significant number of patients due to treatment delay. Another challenge with current CAR-T therapies are that they lead to poor efficacy for most malignancies outside of B cell malignancies such as NHL, acute lymphoid leukemia and multiple myeloma. In addition, even for diseases such as NHL where there are promising initial results, many patients (˜50%) will not have durable remissions after 1 year (6, 7). Though the causes for suboptimal outcomes of CAR-T therapies in patients is likely multifactorial, one challenge is the poor persistence of the infused CAR-T cells in patients. It has been reported that the persistence of the CAR-T cells is correlated to the differentiation status of the manufactured CAR-T product. In particular, more differentiated products are thought to have reduced in vivo persistence as compared to products with more immature/naïve cells (reviewed in (8)).

Traditional CAR-T manufacturing is a long, complex and expensive process that typically involves T cell isolation, T cell activation, T cell transduction (often combined with strategies to enhance transduction efficiency such as spin inoculation, retronectin, polybrene, etc). T cell stimulation to enable efficient transduction typically involves stimulation with CD3 or CD3/CD28 antibodies and cytokine stimulation (ex. IL-2, IL-7, IL-21 and/or IL-15). In addition to utilizing complex and sophisticated manufacturing processes that in many cases requires expensive equipment, the process is time and labor intensive. For example, T cells typically isolated using magnetic beads which involves a long and expensive process. The beads typically also require removal which adds yet another step requiring time, specialized equipment and expertise for the manufacturing. The complex manufacturing also almost always requires the use of a specialized clean room facility to ensure the sterility of the product.

In order for T cells to be efficiently transduced with virus (ex. lentivirus or retrovirus), it has been previously reported and almost universally practiced in the field that the T cells need to be first activated (ex. with CD3 or CD3/CD28 stimulation) in the presence of cytokines (ex. IL-2, IL-7 and/or IL-15) for a period of 1-3 days prior to viral transduction. Therefore, traditional manufacturing involves an initial activation step with CD3 and/or CD3/CD28 followed by 1-3 days before the transduction can efficiently be performed. It is widely practiced for the T cells to be expanded for 1-2 weeks after activation. After viral transduction, traditional manufacturing utilizes a T cell expansion phase partially to generate sufficient T cells for patient infusion.

In a large percentage of cases of clinical CAR-T cell manufacturing, T cells are isolated at the initial manufacturing stages (ie. prior to viral transduction). In particular, a T cell isolation steps are part of all the previously reported rapid genetically engineered T cell manufacturing workflows (1 day or less) that involve viral based genetic engineering. In virtually all cases in which T cells are isolated for clinical manufacturing, T cells are purified using magnetic beads (typically either CD3/CD28 Dynabeads® (ThermoFisher Scientific, Waltham, MA) or CD4/CD8 magnetic beads (Miltenyi Biotec, Bergisch Gladbach, Germany)). These beads which need to largely be removed prior to patient infusion are tightly bound to the T cells and require several days to fall off (typically occurring after internalization of the target surface antigen). For example, the manufacturer ThermoFisher Scientific reports the CD3/CD28 beads take several days to fall off and bead removal will result in a significant fraction of T cells to be lost (due to bound beads) if bead removal is attempted before ˜3 days. (assets.fishersci.com/TFS-Assets/LSG/manuals/11131D_32D_61D.pdf).

Miltenyi Biotec reports their magnetic beads do not release for 2-3 days from bound cells. In fact it has been reported that the Miltenyi Biotec beads instead of actually releasing may get internalized after several days and therefore, they can have unknown impact on the T cell product (thermofisher.com/us/en/home/life-science/cell-analysis/cell-isolation-and-expansion/cell-isolation/see-how-miltenyi-microbeads-interact-with-your-t-cells.html).

Due to the expenses and delay to therapy inherent in the complex manufacturing workflow and benefits of reducing the culture duration of T cells to maintain a naïve population (ex. for enhanced in vivo persistence), a simple and rapid manufacturing workflow is desirable. In particular, a 1 day or less workflow is particularly advantageous as reductions in culture duration would significantly increase the naïve T cell population as well as eventually enable simple, closed system manufacturing outside of a clean room facility. In addition, a process that does not require isolation of T cells would be advantageous for numerous reasons including to increase the simplicity of the process requiring less costs and expertise and decrease the hands on requirements leading to increased scalability. In addition, through avoiding a T cell isolation step the product could contain additional cell types such as NK cells that also have known favorable therapeutic properties such as the ability to lyse tumor or pathogen infected cells. Surprisingly, our studies have also revealed additional benefits of utilizing a mixed population of mononuclear cells (ex. PBMCs or monocyte depleted PBMCs) as opposed to isolated T cells for rapid CAR-T manufacturing. For example, the isolated T cells show a decrease in the highly desirable naïve T cell population in the manufactured product as compared to the same manufacturing process performed using PBMCs without a T cell isolation step.

Prior art clinical rapid CAR-T manufacturing workflows have been reported using viral gene delivery. The manufacturing methods utilize T cell isolation steps which adds an additional layer of unnecessary cost, complexity and can impair the ability to efficiently manufacture cells in the shortest possible time due to inability to remove magnetic beads from cells without significant cell loss.

In one prior art workflow, a <2 day manufacturing protocol was reported that involves ˜24 hr ex vivo culture (ashpublications.org/blood/article/138/Supplement %201/2848/481328/Preservation-of-T-Cell-Stemness-with-a-Novel). This process is reported to involve a T cell isolation step. Key differences are the T cell isolation, method of T cell activation, the use of cytokines and use of automation which adds but significant costs both for the instrument (˜400 k) and high consumable costs). Due to the use of microbeads this product would also likely include internalized microbeads.

Another prior art workflow which reportedly could be performed over 1 day requires the use of CD3/CD28 Dynabeads and high dose IL-2 (300 ul/ml) on isolated T cells. Due to the use of Dynabeads, this process would incur a significant loss of T cells if the cells are harvested at 1 day. In addition, the use of high dose IL-2 can lead to significant differentiation of T cells.

Due to the limitations in the complex, long and expensive manufacturing workflows for genetically modified T cells and the challenges with T cell differentiation with many current approaches, there is a major unmet need to develop rapid, cost-effective, and scalable CAR-T manufacturing platforms that lead to efficacious and affordable CAR-T products. The methods and systems herein meet this long-felt need.

### SUMMARY

In accordance with the purposes and benefits described herein, in one aspect of the disclosure a method for rapid manufacture of a genetically modified T-cell population is provided, comprising steps of obtaining a mixed mononuclear cell population and, substantially simultaneously, activating a T-cell population comprised in the mixed mononuclear cell population and exposing the mixed mononuclear cell population to a viral vector adapted to transduce at least the T-cell population comprised in the mixed mononuclear cell population with a foreign nucleotide. There is no step of T-cell isolation required. In embodiments, the method includes harvesting the mixed mononuclear cell population comprising at least a genetically modified T-cell population at up to 24 hours from the steps of simultaneously activating and exposing to at least one viral vector.

In embodiments, the step of activating is performed by exposing the mixed mononuclear cells to an activation agent selected from one or more of the group of cytokines consisting of IL-2, IL-7, IL-15, and IL21, and/or to an activation agent selected from activators for one or more of CD3, CD28, OX40, CD2, CD27, ICAM-1, LFA-1 (CD11a/CD18), ICOS (CD278), and 4-1BB (CD137). In embodiments the activating is performed by exposing the mixed mononuclear cells to an activation agent selected from one or more of the group consisting of IL-7 and IL-15. In other embodiments, the method includes a step of at least partial depletion by adherence of a monocyte population comprised in the mononuclear cell population prior to the steps of activating and exposing to the viral vector.

In embodiments of the method, the steps of activating and exposing to the viral vector are preferably performed in the absence of any exogenous cytokine. The step of activating may be performed by exposing the mixed mononuclear cell population to one or more of a CD3 activator, a CD28 activator, soluble or surface-bound CD3 antibody, or soluble or surface-bound CD28 antibody.

The mixed mononuclear cell population comprising the genetically modified T-cell population. The mixed mononuclear cell population may be obtained by apheresis or a peripheral blood draw.

In embodiments, the viral transduction vector is selected from the group consisting of a lentivirus, a retrovirus, and an adenovirus. In embodiments, a step of differential centrifugation is provided following the step of substantially simultaneously activating and exposing to the viral transduction vector to remove a plasmid DNA from genomic DNA by DNA size selection.

In another aspect, the present disclosure provides a genetically modified T-cell, produced by the above method.

In yet another aspect, the present disclosure provides a closed system kit for performing the method for rapid manufacture of a genetically modified T-cell population according to the above method. The closed system kit may include a first sterile vessel adapted for receiving a mixed mononuclear cell population, a second sterile vessel adapted for receiving a mixed mononuclear cell population depleted of monocytes, a bead-free T-cell activation agent, a viral vector adapted to transduce a T-cell population with a foreign nucleotide, a suitable culture media, and a suitable cell washing solution. In embodiments, at least the first vessel is fabricated of a material suitable for depletion of monocytes from the mixed mononuclear cell population. The first and second vessels may be adapted for sterile introduction of the bead-free T-cell activation agent, the viral transduction vector, the culture media, and the cell washing solution.

The various elements/reagents of the closed system kit are otherwise substantially as described above. One or both of the first and the second vessel may be selected from the group consisting of a cell culture bag and a cell culture flask.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1A shows a comparison of transduction efficiencies using a 20 hr simultaneous transduction/activation process and GFP lentiviral vector using different T cell activation reagents on monocyte depleted PBMCs. After 20 hr, the cells were washed to remove free virus/activation reagent and the Cloudz® reagent was removed with dissolution buffer. GFP expression was determined by flow cytometry 3 days after transduction/activation. The workflow utilized is described in the section entitled Detailed example of rapid manufacturing workflow. Panel A depicts the use of 3 commercial reagents that all involve CD3 and CD28 antibodies conjugated to a matrix or beads.

FIG. 1B shows T cell manufacturing performed as in FIG. 1A with the exception of use of soluble CD3 (100 ng/ml OKT3) or a combination of soluble CD3 (100 ng/ml) and CD28 (300 ng/ml) antibodies as the activation reagent. Further, the manufacturing was performed in the absence or presence of IL-7 and IL-15.

FIG. 1C shows T cell manufacturing performed as described in FIG. 1B with the exception of use of Immunocult® at the manufacturers recommended concentration, included as a T cell activation reagent and cytokine was used for all samples (TL-7 and IL-15).

FIG. 1D shows T cell manufacturing performed using the workflow described in FIG. 1A with the exception that T cell activation was performed using soluble CD3 (100 ng/ml OKT3), surface bound CD3 (surface coated with 5 μg/ml OKT3) or TransAct® (at the manufacturers recommended concentration). CD69 expression was measured by flow cytometry at product harvest (20 hour culture).

FIG. 2 illustrates simultaneous vs sequential T cell activation: CD19 CAR expression was assessed using an anti-FMC63 antibody (AcroBiosytems, Newark, Delaware) by flow cytometry. The cells were examined for CAR expression 4 days after transduction. PBMCs were plated and activated with the Cloudz® T cell activation reagent at either the same time as lentiviral vector addition or 24 hours prior to virus addition.

FIG. 3 shows a comparison of various cytokines on modulating the transduction efficiency using the rapid manufacturing workflow. Monocyte depleted PBMCs were transduced with CD19 CAR lentiviral vector using the process described in the section Detailed example of rapid manufacturing workflow using the indicated cytokines and assessed for CD19 CAR expression after 72 hours by flow cytometry using an FMC63 specific antibody (Acrobiosystems).

FIG. 4 presents an assessment of different culture durations using the rapid manufacturing workflow. Monocyte depleted PBMCs were activated and transduced with CD19 CAR vector as described in the section Detailed example of rapid manufacturing workflow. The cells were washed to remove free virus and the CloudZ® T cell activation reagent was removed at 6 or 17 hours after initiation of the process. The cells were then maintained in culture for 72 hours in order to assess CD19 CAR surface expression by flow cytometry.

FIG. 5 shows that UF-KURE19 cells demonstrated increased in vivo efficacy over similar CAR-T cells manufactured for 6 days. NSG mice (n=5-7 per group) were injected with RAJI-luciferase cells (0.5×10⁶) intravenously and then on day 7 with the indicated number of UF-Kure19 CAR-T cells or CAR-T cells expressing the same CAR manufactured for 6 days.

FIG. 6 shows that exogenous cytokine is not needed for an effective rapid T cell manufacturing workflow. NSG mice were injected with Raji-luciferase cells and the indicated CD19 CAR-T products or vehicle were injected after 7 days followed by bioluminescence imaging on the indicated dates.

FIG. 7A shows testing of qPCR release testing with low fragment removal. The data depict the results of TaqMan based qPCR testing of VSVG (replication competent lentivirus) on DNA samples prepared from CD19 CAR-T transduced cells using the rapid manufacturing workflow and harvested after 20 hours. DNA preparation was performed using the protocol described above (using the PacBio reagent) for low fragment removal. The PCR testing was performed on samples with and without low fragment removal.

FIG. 7B shows data depicting the vector copy number per transduced cell results for T cells transduced with CD19 CAR lentiviral vector and cultured for 8 days at various MOI's. The CD19 CAR expression was determined by flow cytometry using a CD19 CAR specific antibody (AcroBiosystems) and the copy number was determined by qPCR for GAG and PTPB2.

FIG. 7C shows data depicting the vector copy number per transduced cell and CD19 CAR surface expression for T cells manufactured using the rapid manufacturing workflow using an MOI of 10:1 for the vector described in FIG. 7B. The vector copy number per transduced cells was determined by qPCR and flow cytometry as described in FIG. 7B. The low fragment size DNA was removed prior to PCR using the SRE kit (PacBio).

FIG. 7D shows the results of DNA gel electrophoresis demonstrating that the low fragment removal does not significantly impact the amount or size distribution of the genomic DNA. Genomic DNA from two different T cell samples that were lentivirally transduced with CD19 CAR vector and harvested after 20 hours were prepared using the DNeasy Blood and Tissue Kit. Agarose gel electrophoresis was either performed directly on the genomic DNA samples or was performed after performing low input depletion using the PacBio 10 kb SRE Kit. Lane 1: DNA marker Lane 2: Sample 1 Total Genomic DNA Lane 3: Total Genomic DNA after SRE kit processing Lane 4: Sample 2 Total Genomic DNA Lane 5: Sample 2 Total Genomic DNA after SRE kit processing.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

To address the above-summarized and other shortcomings in prior art methodology and in order to develop a robust, cost effective, simple and scalable ultra-fast T cell manufacturing method (i.e., 1 day or less), we developed methods to manufacture CAR-T cells without the use of magnetic beads or in some cases without even the need for T cell isolation or the use of cytokines. In this case we surprisingly identified a method that enables the high transduction efficiency of T cells (starting with either mixed PBMCs, monocyte depleted PBMCs or isolated T cells) using concurrent T cell activation and viral transduction without the need for any enhancers (ex. polybrene, spin inoculation, retronectin). Surprisingly, this process can also be conducted in the absence of any cytokines (Ex. IL-2, IL-7 and/or IL-15) or in the presence of cytokines. Contrary to previous suggestions, the T cell transduction efficiency using this method was found to be as high as the transduction efficiency utilizing the more traditional T cell activation followed by viral transduction 1-3 days after activation. Of note another advantage of using PBMCs as a starting source as opposed to isolated T cells is that ability to utilize a CD3 activation alone without the requirement of CD28 co-stimulation due to the presence of other mononuclear cells that provide stimulatory signals. It has known that when T cell activation is performed, that CD3 stimulation alone without the necessity of using CD3/CD28 activation reagents, there is more preservation of desirable central memory T cells (9). Therefore, the developed T cell manufacturing workflow involves a highly simplified process that can significantly reduce costs and improve efficiency as compared to previously reported methods.

Development of an Ultra-Fast Manufacturing Process that can Utilize PBMCs, Monocyte Depleted PBMCs or Isolated T Cells as a Starting Product

According to the presently disclosed methods, the manufacturing can occur with or without the requirement for a T cell pre-isolation step. By not performing a T cell pre-isolation step, the manufacturing workflow can be performed more rapidly and for a reduced cost and surprisingly also yields a product with a higher percentage of desirable naïve T cells. If a blood apheresis or peripheral blood sample is used without direct T cell isolation, the T cells can be enriched through monocyte depletion by simple adherence to a solid surface (ex. tissue culture flask/plate or bag). The monocyte depletion can be performed on an adherent surface such as on a plate or bag and can also be performed in a closed system. Monocyte depletion is a simple, cheap and rapid way to partially purify the product.

In another aspect, by avoiding performing a T cell isolation step, the final product can include not only genetically engineered T cells but also other cell types such as NK cells that may exhibit beneficial therapeutic properties.

According to the present disclosure, T cells are simultaneously activated using in one aspect a bead-free activation reagent and transduced in the presence of low concentrations of IL-7 and IL-15 (ex. 5-10 ng/ml or lower) importantly in the absence of IL-2. IL-2 is a cytokine that is known to drive T cell differentiation and cells cultured in the presence of IL-2 are known to show reduced preservation of their naïve/undifferentiated phenotype. This is particularly true when high doses (ex. 300 IU/ml) are utilized (11-12).

In another aspect, surprisingly it has been found that workflow involving the simultaneous activation and transduction of T cells is highly efficient in the presence of either IL-7 or IL-15 and does not require both cytokines. Therefore, the manufacturing workflow can be performed with IL-7 alone or IL-15 alone.

In another aspect, surprisingly, the workflow involving the simultaneous activation and transduction of T cells is highly efficient in the complete absence of exogenous cytokine addition including IL-2, IL-7 or IL-15. Therefore, the manufacturing workflow can be performed with the addition of any exogenous cytokine.

In another aspect, the genetically modified T cell product can be manufactured in less than 24 hours, preferably including a culture time of approximately 17-20 hours.

In this workflow, there is no requirement for a pre-activation step prior to viral transduction or T cell isolation both of which saves critical manufacturing time and costs. There are no previous reports that rapid CAR-T manufacturing can be efficiently performed using simultaneous transduction and activation of non-purified mononuclear cells (ex. PBMCs) or monocyte deleted PBMCs (as opposed to purified T cells).

The cell activation can occur using various reagents that activate T cells through CD3 or CD3 and CD28 including Transact (MiltenyiBiotec), Cloudz T cell activator (Biotechne), soluble or surface bound CD3 and/or CD28 antibodies or custom microbubbles with conjugated CD3 and/or CD3/CD28 antibodies. In particular, the Cloudz T cell activator is found to enable a high and unexpected ability to simultaneously activate and virally transduce T cells as compared to Transact. In addition, soluble or surface bound CD3 with or without CD28 also functions will in the less than 1 day workflow described here.

The cell activation can also occur using reagents that activate T cells through CD3 and other co-stimulatory molecules besides CD28 (or in addition to CD28) such as OX40, CD2, CD27, ICAM-1, LFA-1 (CD11a/CD18), ICOS (CD278) and 4-1BB (CD137).

After activation and transduction of the cells, the cells can be harvested in less than one day after transduction (ex. ˜17-20 hr) and can be used directly for therapeutic purposes or cryopreserved for later use. This method of manufacturing leads to T cells that exhibit high therapeutic efficacy despite the fact that the expression of the gene transduced into the T cells is not fully expressed when the cells are harvested as well as infused to the recipient.

Therefore, this method enables a rapid manufacturing protocol for cell therapy products that can be performed in less than 1 day.

In another component of this approach, the T cell manufacturing process can be performed in a fully closed system using a manual or automated process. In another component the manufacturing can be performed in a fully closed system outside of a clean room allowing manufacturing at many centers without specialized clean room infrastructure.

Another component of this approach is a method to eliminate false positive reactivity for required release testing of virally transduced cell therapy products that occurs with rapid manufacturing. Residual plasmid DNA from cell transfection (Ex. 293 or 293T cells) to produce lentivirus or retrovirus is present in cell therapy products during the first several days of manufacturing. This plasmid DNA gives false positive results for vector copy number and replication competent virus testing qPCR assays. In this workflow, release testing is integrated into the workflow and involves a size separation step based on differential centrifugation to eliminate this plasmid DNA and enable the clinical use of the rapidly manufactured T cell products that is often required for product release.

The presently-disclosed subject matter is further illustrated by the following specific but non-limiting examples. The following examples may include compilations of data that are representative of data gathered at various times during the course of development and experimentation related to the present invention.

### Examples

### Development of Rapid Genetically Modified T Cell Manufacturing Process

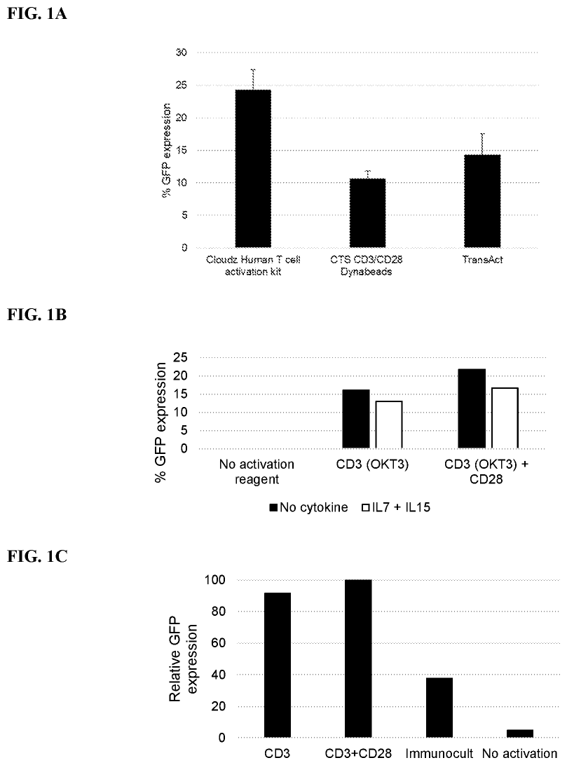

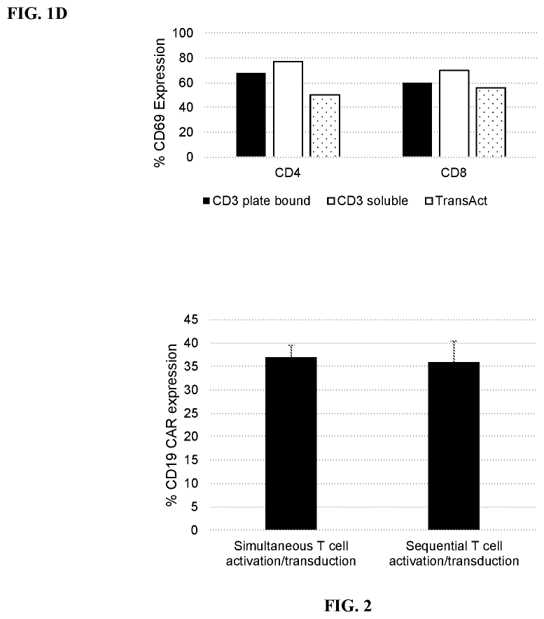

To date the previously described rapid (ex. 1 day or less) virus-based gene modified T cell manufacturing processes involve an initial T cell isolation step. Here we developed a rapid manufacturing process that can utilize PBMCs or monocyte depleted PBMCs in addition to isolated T cells. An optional initial step if PBMCs are going to be utilized as a starting source is to perform monocyte depletion as in some cases it is desirable to partially deplete monocytes from the product. This depletion will for example enable virus to preferentially infect the specific cell types that are more desirable for the final product including T cells and in some cases NK cells. However, the depletion is not complete which will allow monocytes to provide stimulatory signals (ex. CD80/CD86) to the T cells (ex. enable T cell activation using exogenous CD3 stimulation alone without the necessity for co-stimulation with exogenous CD28 or other stimuli). In general, the depletion method described below leads to an approximately 50% reduction in monocytes from the starting blood product starting at 2 hours after plating as seen in FIG. 1. Additional duration of incubation after 2 hours did not significantly impact the depletion using this approach. In addition, the cell density at which cells where plated (5×10⁶ cells/ml and 2×10⁶ cells/ml) demonstrate equivalent results. The depletion shows similar results utilizing tissue culture plates or flasks or in a closed system using bags such as the VueLife® “AC” Series bags manufactured by Saint Gobain (Paris, France).

TABLE 1
Depletion of monocytes from PBMCs.

| | 1 hr | 2 hr | 3 hr | 4 hr |
|---|---|---|---|---|
| Cell Concentration Plated | MO % | MO % | MO % | MO % |
| 2 × 10⁶/ml | 21.46 | 13.77 | 14.86 | 14.95 |
| 5 × 10⁶/ml | 20.87 | 11.25 | 10.97 | 11.09 |

Mononuclear cells from a peripheral blood apheresis sample were incubated in a 6 well tissue culture plate in a tissue culture incubator at 37 degrees Celsius for the indicated timepoints. The percent monocytes was determined in the non-adhered cells at the following timepoints using a hematology analyzer (Hemavet®, Drew Scientific, Miami Lakes, Florida). The starting population consisted of a starting monocyte percentage of 25.02%. MO %=monocyte percentage.

If isolated T cells are to be used then they can be isolated directly from whole blood, an apheresis sample or another source of T cells. The T cells can be isolated using any available method. For example a magnetic bead approach such as CD3, CD4 or CD8 magnetic beads (ex. Miltenyi Biotec microbeads or ThermoFisher Scientific Dynabeads®) can be used. The T cells can also be efficiently and rapidly isolated using CD3 microbubbles, CD3/CD28, or microbubbles conjugated with CD3 and any other T cell co-stimulatory ligand. In particular microbubbles, particularly lipid microbubbles provide a rapid and efficient method of isolation and simultaneously also provides an activation signal.

The activation of the T cells can be accomplished through multiple approaches. If PBMCs or monocyte PBMCs are utilized, a soluble activation reagent or surface bound (ex. plate/bag) is preferred for the rapid manufacturing workflow over magnetic bead based approaches. One effective activation approach is to use CD3/CD28 dissolvable microspheres (Cloudz® Human T cell activation reagent from Biotechne/R&D Systems (Minneapolis, Minnesota). This reagent consists of an alginate copolymer that is dissolvable within minutes and therefore does not require magnetic beads and it does not lead to contamination of the product. Alternative activation reagents can also be utilized such as Immunocult® CD3/CD28 Activator (Stem Cell Technologies, Vancouver Canada). Alternative activation agents that can be utilized include CD3 or CD3/CD28 magnetic beads (ex. Dynabeads® ThermoFisher Scientific) or TransAct® (Miltenyi Biotec). The magnetic beads based and TransAct® agents are not considered as optimal as the magnetic beads require increased labor and a removal step that is challenging due to tightly bound beads at early timepoints. This may lead to excessive loss of cells. The TransAct® activation reagent is also reported to be a “gentler and slower” activation reagent as compared to the other products which leads to an optimal window of viral transduction at later timepoints. TransAct® has been reported to require 1-2 days for T cell activation prior to viral transduction in order to enable optimal viral transduction. In addition, it is reported that if TransAct® is washed away prior to 2-3 days after addition it can hamper T cell proliferation (miltenyibiotec.com/upload/assets/IM0017348.PDF). Therefore, TransAct® is not the preferred activation reagent for a manufacturing protocol that involves simultaneous activation/transduction and harvest of the product in less than one day.

In addition to matrix associated activation reagents, soluble or surface coated CD3 antibody (ex. OKT3 antibody or other CD3 antibodies) either alone or in combination with soluble CD28 antibody or other T cell co-stimulatory stimuli is another effective activation strategy that is fully compatible with the described rapid manufacturing workflow. In particular, this approach benefits from the PBMC based approach which enables activation of the T cells without the need for exogenous CD28 or other agents for co-stimulation. In addition, this method of using CD3 antibody such as OKT3 leads to significantly reduced costs as compared to other activation stimuli and minimizes risks present with scaffolds (ex. alginate, magnetic beads etc) present in the manufactured product that can have unknown side effects in patients.

Specific T cell activation reagents have been found to function more efficiently using the less than 1 day rapid manufacturing workflow described here. In order to compare specific T cell activation reagents, monocyte depleted PBMC's (200,000 cells in 100 μl) were plated in a 96 well plate in 3% CTS® immune cell serum replacement (ThermoFisher Scientific) in TexMACs® (Miltenyi Biotec) media containing IL-7 (10 ng/ml) and IL-15 (5 ng/ml). GFP lentiviral vector and the indicated activation reagent (using the concentrations suggested by the manufacturer) were added and the cells were cultured for 4 days. The cells were washed and media was changed after 20 hr to remove the free virus and free activation reagent. For the cells containing the Cloudz® regeant, the dissolution buffer was utilized to also remove the activation reagent. The expression of GFP was measured by flow cytometry. As seen in FIG. 2A, the Cloudz® human T cell activation reagent was more effective as compared to TransAct® and CTS CD3/CD28 Dynabeads® in maximizing transduction efficiency using the rapid manufacturing workflow. Of note for the majority of figures, the amount of viral vector employed was kept low to easily facilitate differences between conditions as opposed to using excess virus to maximize transduction efficiency. In FIG. 2, the ability of the rapid manufacturing workflow to lead to high transduction efficiencies can be observed using high levels of lentiviral vector (though still maintaining a vector copy number per transduced cell of less than 5). As also shown in FIG. 2B, the rapid manufacturing workflow also works with soluble CD3 and/or soluble CD3 and CD28 antibodies (FIG. 2C). For example, the use of the widely used CD3 antibody, OKT3, is possible with this workflow (FIG. 2C). Using the manufacturing process with monocyte depleted PBMCs, the use of CD3 alone as compared to CD3 and CD28 antibody supplementation functions similarly in terms of transduction efficiency. Of note, OKT3 CD3 antibody stimulation functioned more efficiently than Immunocult® in terms of transduction efficiency in this manufacturing workflow (FIG. 2C). Using the rapid manufacturing workflow, both surface bound (ex. plate or flask) CD3 antibody and soluble CD3 antibody lead to similar activation of CD4 and CD8 T cells as measured by CD69 expression at the product harvest (20 hours after culture initiation). Of note, it was observed that Transact® led to a reduced level of CD69 upregulation in CD4 T cells as compared to the groups treated with soluble/surface bound CD3. This finding shows that either soluble CD3 or CD3 bound to the culture container can be used efficiently for T cell activation using the rapid workflow. Further these activation reagents appear superior to other agents such as TransAct®.

It has been generally assumed in the field that sequential T cell activation followed by viral transduction is needed to maximize the viral transduction efficiency. However, the results here demonstrate that at least utilizing our specific manufacturing workflow, that simultaneous T cell activation and viral transduction leads to essentially equivalent transduction efficiencies as the sequential method. For example, monocyte depleted PBMCs were activated using the Cloudz® T cell activation reagent and either transduced with lentiviral CD19 Chimeric Antigen Receptor (CAR) vector at the same time of Cloudz® addition or 24 hr later using the manufacturing process described in the section Detailed example of rapid manufacturing workflow. As can be seen in FIG. 2, there is no significant difference between CAR expression using simultaneous or sequential viral transduction using the described manufacturing workflow.

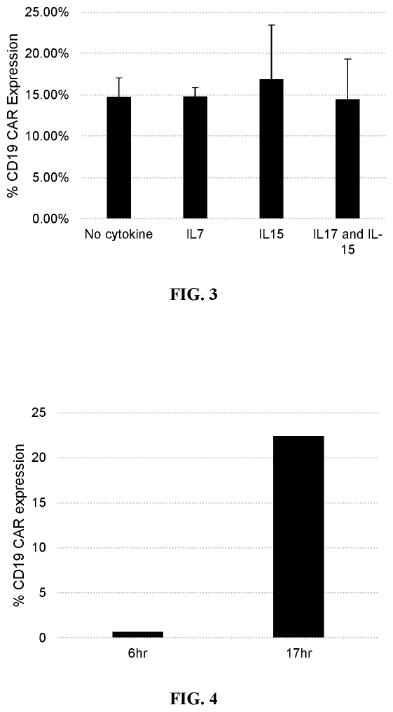

Both traditional and previously reported rapid T cell manufacturing approaches use culture conditions that almost universally employ cytokines such as IL-2, IL-15 and/or IL-7. To evaluate the optimal cytokine for use in the rapid manufacturing workflow, the manufacturing process described in the section Detailed example of rapid manufacturing workflow was performed using IL-2 (300 u/ml), IL-15 (5 ng/ml), IL-7 (10 ng/ml), a combination of IL-15 and IL-7 or a complete absence of exogenous cytokine (FIG. 4). There was no difference observed in transduction efficiency observed when comparing the expression of CD19 CAR in T cells at 72 hours after transduction among the different cytokine groups. Surprisingly, there was also no difference in the transduction efficiency in the complete absence of cytokine. Therefore, the manufacturing process can be further simplified to be performed in the absence of any exogenous cytokine to further reduce costs and to prevent stimulatory effects on the T cells that may lead to undesirable differentiation/activation. As seen in FIG. 1, the rapid T cell manufacturing was also nearly as efficient in leading to GFP transduction of T calls when comparing manufacturing performed with and without cytokine.

Another key attribute of the manufacturing process is the culture duration. As one goal of the manufacturing workflow is to limit its duration to preserve the naïve T cell fraction, the manufacturing process was compared utilizing a 6 hour and 17 hour culture duration. At the end of both of these timepoints, the Cloudz® reagent was removed using the dissolution buffer, the cells were washed to remove virus and suspended in fresh media without virus or activation reagent for a total of 72 hours of culture in order to measure CAR expression. As seen in FIG. 4, a 6 hour culture duration of lentiviral vector and the T cell activation reagent was insufficient to lead to significant CD19 CAR expression as measured at 3 days by flow cytometry. Intermediate durations greater than hours and less than around 15 hours is not viewed as preferable due to limitations of being able to perform the manufacturing during typical work day hours.

As the manufacturing platform can utilize PBMCs or monocyte depleted PBMCs as a starting source and the product is cultured less than one day, the final product will consist of additional mononuclear cells in addition to T cells. To assess the composition of the product, 3 manufacturing runs were performed using the method described in the section Detailed example of rapid manufacturing workflow. As seen in Table 2, T cells are the largest fraction of the product, but the product also contains smaller numbers of B cells, NK cells and monocytes. As a preferred method for the workflow involves the production of a cryopreserved product, this testing was performed after thaw of the product. As granulocytes represent a small component of the starting aphersis product and also are highly susceptible to freeze/thaw, there were virtually no granulocytes detected in the product.

TABLE 2
Product composition from 3 manufacturing runs on
thawed UF-KURE19 product (17 hr manufacturing).

| | Product 1 | Product 2 | Product 3 |
|---|---|---|---|
| % Viability | 88 | 94 | 90 |
| % T cells (CD3) | 78 | 82 | 78 |
| % CD4 of T cells | 53 | 45 | 56 |
| % CD8 of T cells | 47 | 55 | 44 |
| % B cells (CD19) | 7 | 5 | 5 |
| % NK cells (CD56) | 11 | 6 | 8 |
| % Monocytes (CD14) | 6 | 3 | 7 |

Product composition was determined using flow cytometry with CD3, CD4, CD8, CD19, CD56, and CD14 antibodies as well as 7-AAD to assess viability.

As significant expression of proteins such as CARs after lentiviral transduction does not occur within 17-20 hours after in the T cells, in order to assess the activity of a transduced CD19 CAR-T product in vitro, we manufactured CD19 CAR-T cells from monocyte depleted PBMCs using the manufacturing workflow described in the section Detailed example of rapid manufacturing workflow. After 20 hr of activation/transduction, the cells were washed of free virus and the CloudZ® T cell activation reagent was removed. The cells were then cultured for a total culture period of 3 days and then assessed for cytotoxic activity against target RAJI human lymphoma cells and CAR surface expression was measured. As seen in Table 3, the rapid manufactured CAR-T cells were able to efficiently lyse RAJI tumor cells.

TABLE 3
Results of testing cytotoxic activity in vitro of rapid
manufactured CD19 CAR-T cells after 3 days of culture.

| | Product 1 | Product 2 | Product 3 |
|---|---|---|---|
| % CAR expression on T cells | 50 | 44 | 53 |
| % Lysis of RAJI cells | 51 | 56 | 56 |
| (5:1 CAR-T/RAJI ratio) | | | |

Monocyte depleted PBMCs were transduced with CD19 CAR lentiviral vector and T cells were activated with the CloudZ® T cell activation reagent as described in the section Detailed example of rapid manufacturing workflow. The free virus was removed and T cell activation reagent was dissolved after 20 hr and the cell culture was continued for 3 days. The cytotoxic activity of the CD19 CAR-T cells was assessed against RAJI tumor cells by measuring the loss of calcien AM dye from the tumor cells after a 4 hour co-culture with CAR-T cells by flow cytometry. The CD19 CAR expression was measured by flow cytometry using an FMC63 specific antibody (AcroBiosystems).

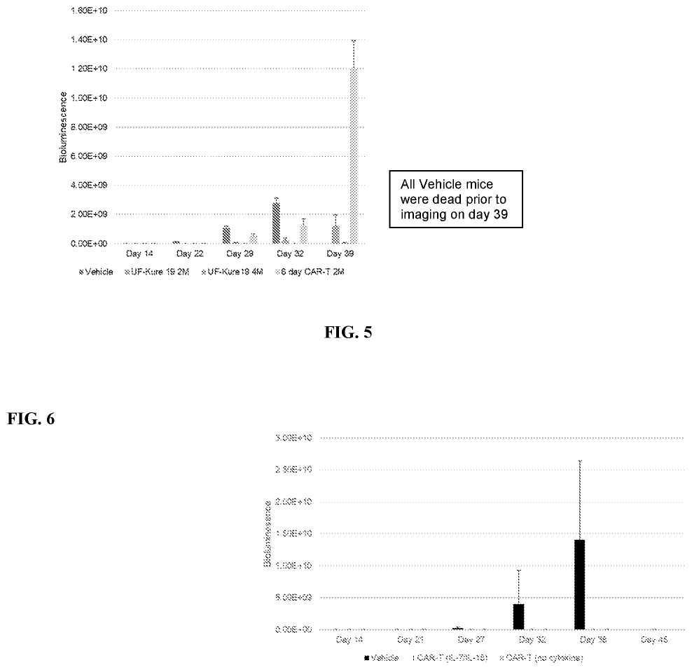

As the CD19 CAR protein is not significantly expressed on the surface of T cells using standard flow cytometric methods when the product is harvested at 17-20 hours of culture, the activity of the T cells was also evaluated in mouse models to demonstrate the efficacy of the product. In this manner, the full expression of the CAR occurs in vivo and subsequently enable the T cells to attain their cytotoxic activity against cells expressing human CD19. To demonstrate the improved efficacy of the rapid manufactured CAR-T product, this in vivo studied utilized cryopreserved cells that were manufactured for 17 hours starting with monocyte depleted PBMCs and transduced with CD19 CAR lentiviral vector following the workflow described in the section Detailed example of rapid manufacturing workflow. This product that expresses the CD19 CAR was termed UF-KURE19 cells. Cells that were manufactured using the same workflow but were maintained in culture for 6 days instead of 17 hours were termed Kure19. The cells were tested in a circulating mouse model of human lymphoma that involves the injection intravenously of human RAJI tumor cells into immunodeficient mice (NSG) followed by the intravenous injection of a single dose of CAR-T product 7 days after tumor cell injection. Though traditionally, ˜5 million CAR T cells are used in this model to demonstrate significant efficacy, lower doses were utilized due to an expected increase in potency of the rapid manufactured product. In this case for the UF-Kure19 product, doses of 2 and 4 million CAR positive T cells were utilized for the UF-KURE19 cohorts and 2 million CAR positive T cells for the 6 day manufactured product. As can be seen in FIG. 5, UF-KURE19 cells demonstrate marked efficacy at both the low and high dose level. In contrast, while the 6 day cultured product demonstrates reduced tumor progression as compared to vehicle treated mice, the efficacy is dramatically reduced when compared to the UF-KURE19 product. Similar to the mouse studies employing Kure19, UF-KURE19 injected groups tolerated the therapy well (as monitored by weight change, feeding, appearance and behavior) with no obvious signs of toxicity.

CD19 CAR-T cells have been found to persist (as measured by detection of the transgene) for months and even years. For example, the Tisa-cel product has been found to persist for at least 2 years in some patients that have had favorable clinical outcomes (10). The proliferation of human T cells in the blood of RAJI lymphoma tumor bearing NSG mice was measured by flow cytometry. In FIG. 9, the average number of human T cells detected per microliter of mouse peripheral blood is shown at the indicated time points after RAJI tumor cell injection (0.5 million i.v.) into female NSG mice (measurements performed on n=3-5 mice per time point). Of note, the blood samples for these measurements were derived from the mouse efficacy study shown above. As shown in Table 4, the efficacy of the UF-KURE19 cells is significantly higher than the 6 day manufactured KURE19 CAR-T cells which correlates with human T cell expansion.

TABLE 4
T cells detected by flow cytometry per microliter of mouse blood

| | UF-Kure19 | UF-Kure19 | 6 Day CAR-T |
|---|---|---|---|
| | 4 × 106 cells | 2 × 106 cells | 2 × 106 cells |
| Day 32 | 126.7 | 60.1 | 2.7 |
| Day 39 | 410.1 | ND | 7.3 |
| Day 46 | 969.1 | 995.6 | 14.9 |

ND = Not determined

As described above, the use of cytokine during the rapid manufacturing workflow was not necessary to achieve significant transduction efficiency. Therefore, the impact of the use of cytokine on the memory/differentiation status of the T cells at harvest (20 hours) was assessed by flow cytometry. In addition, this study employed the use of different cytokines in the manufacturing process as noted in FIG. 10 or the absence of cytokine to better assess the impact of cytokine on the product. Finally this study also compared monocyte depleted PBMCs and the use of isolated T cells as a starting source. The manufacturing workflow described in the section Detailed example of rapid manufacturing workflow was utilized except that purified T cells were used as a starting source for a subset of the samples. T cells were purified from peripheral blood using the RosetteSep® Human T cell Enrichment kit (StemCell Technologies). As seen in Table 5, in CD8+ T cells when comparing product that was manufactured starting with isolated T cells as compared to monocyte depleted PBMCs, it is observed that the CD8+ T cell component of the product is not identical. In particular, when starting with PBMCs there is a slightly lower level of effector memory T cells and a slightly higher percentage of TEMRA cells. The role of the CD8+ TEMRA cells is not fully elucidated but they are thought to impart increased cytotoxic activity that may be beneficial. The level of the highly beneficial naïve T cells that are known to correlate with CAR-T efficacy is similar starting with PBMCs or isolated T cells in the CD8+ T cell compartment, however, there is a higher percent of the beneficial central memory T cells in the PBMCs as compared to isolated T cells and lower level of the more differentiated and less desirable effector memory cells. In the CD4+ T cell compartment, there is an increase in the highly beneficial naïve T cell percentage when starting with PBMCs as compared to isolated T cells. Therefore, based upon phenotyping the product, it is found that starting the workflow using PBMCs imparts a more preferable workflow that may be predicted to lead to improved clinical outcomes and greater CAR-T cell persistence in vivo. In addition, this study also investigated the use of different cytokines (IL2, IL7, IL-15) or the absence of cytokine during manufacturing. As seen in Table 5, there were no significant differences during the 20 hour workflow in any conditions in terms of the T cell memory/differentiation phenotype when starting with PBMCs further suggesting that surprisingly cytokine is not a necessary component for this manufacturing workflow when starting with monocyte depleted PBMCs. Interestingly, when starting with isolated T cells there was a reduction in naïve T cells noted when no cytokines were employed suggesting that the use of cytokines may be more important for manufacturing when employing isolated T cells as a starting source. It is possible that the PBMCs provide an endogenous source of cytokines.

TABLE 5
Phenotype of T cells manufactured using the rapid workflow.

| CD8+ T cells | | TEMRA | naïve | CM | EM |
|---|---|---|---|---|---|
| Activation | Cytokine | CCR7−CD45RA+ | CCR7+CD45RA+ | CCR7+CD45RA− | CCR7−CD45RA− |

T cells

| | | | | | |
|---|---|---|---|---|---|
| No | No | 4.79 | 63 | 6.01 | 26.2 |
| Yes | No | 5.87 | 58.9 | 8.53 | 26.7 |
| Yes | IL2 (300 u/ml) | 6.75 | 65.7 | 5.81 | 21.7 |
| Yes | IL7 (10 ng/ml) | 6.73 | 62.1 | 6.6 | 24.6 |
| Yes | IL15 (5 ng/ml) | 5.02 | 66.1 | 6.13 | 22.8 |
| Yes | IL7 + IL15 | 5.62 | 65.4 | 6.86 | 22.2 |

PBMCs

| No | No | 3.3 | 66.8 | 8.2 | 21.8 |
|---|---|---|---|---|---|
| Yes | No | 10.4 | 64.8 | 10.1 | 14.8 |
| Yes | IL2 (300 u/ml) | 10 | 64.7 | 12 | 13.3 |
| Yes | IL15 (5 ng/ml) | 8.2 | 66.3 | 11.6 | 13.9 |
| Yes | IL7 + IL15 | 5.6 | 68.4 | 11.8 | 14.2 |
| CD4+ T cells | | TEMRA | naïve | CM | EM |
| Activation | Cytokine | CCR7−CD45RA+ | CCR7+CD45RA+ | CCR7+CD45RA− | CCR7−CD45RA− |

T cells

| | | | | | |
|---|---|---|---|---|---|
| No | No | 4.1 | 66.1 | 18.1 | 11.6 |
| Yes | No | 3.5 | 63.6 | 23.2 | 9.7 |
| Yes | IL2 (300 u/ml) | 4.1 | 70.3 | 18.3 | 7.3 |
| Yes | IL7 (10 ng/ml) | 3.3 | 68.0 | 20.4 | 8.3 |
| Yes | IL15 (5 ng/ml) | 1.9 | 70.1 | 21.2 | 6.8 |
| Yes | IL7 + IL15 | 2.4 | 70.2 | 20.6 | 6.8 |

PBMCs

| | | | | | |
|---|---|---|---|---|---|
| No | No | 2.0 | 74.4 | 15.9 | 7.8 |
| Yes | No | 5.4 | 77.0 | 13.8 | 3.8 |
| Yes | IL2 (300 u/ml) | 4.7 | 79.7 | 12.4 | 3.2 |
| Yes | IL15 (5 ng/ml) | 3.8 | 78.7 | 14.3 | 3.2 |
| Yes | IL7 + IL15 | 2.7 | 82.0 | 12.3 | 3.1 |

T cells were manufactured using the rapid workflow described in the section Detailed example of rapid manufacturing workflow or using the identical workflow except isolated T cells were used as a starting cell source. After 20 hours the cells were assessed for T cell phenotype by flow cytometry.

As the in vitro studies demonstrated that cytokine does not appear to be necessary to efficiently transduce T cells or maintain a preferable memory/differentiation T cell phenotype using the rapid manufacturing process when starting with PBMCs, mouse in vivo efficacy studies were performed to further confirm these findings. CD19 CAR-T cells were manufactured using the rapid manufacturing workflow starting with monocyte depleted PBMCs using either IL-7 (10 ng/ml) and IL-15 (5 ng/ml) or no cytokine during the culture. The same human lymphoma tumor model (RAJI) in NSG mice described above was employed. In this case 1.2×10⁶ CD19 CAR positive T cells were injected per mouse 7 days after tumor cell injection. As seen in FIG. 6, the CAR-T product manufactured in the complete absence of exogenous cytokine efficiently controlled tumor progression in a similar fashion as the product manufactured with cytokine. All vehicle control mice were dead from disease progression prior to the imaging performed on day 45. Of note, the low dose of 1.2×10⁶ CD19 CAR positive T cells showed high efficacy as shown in FIG. 6A further demonstrating the potency of the rapid CAR-T product. This study also clearly demonstrates exogenous cytokine is not needed to create a rapid (less than 1 day) manufactured CAR-T product starting from PBMCs.

In addition to similar efficacy, there was no decrease in circulating human T cells in the mice when the cytokine free product was utilized. Human T cells were quantified in the mouse blood from the experiment shown in FIG. 6 on day 41 after tumor cell injection using a human specific CD3 antibody and flow cytometric analysis. No cytokine CAR-T group: 4495 human T cells per microliter mouse blood. IL7/IL15 CAR-T group: 1618 human T cells per microliter mouse blood.

### Detailed Example of Rapid Manufacturing Workflow.

Selected representative modifications to this workflow are shown in parenthesis. In addition to this relatively manual workflow presented below, the entire workflow can be performed in a fully closed and/or semi-automated or automated fashion. For example, cell washing, harvesting etc. can be performed using automated equipment.

Manufacturing Process to Produce Less than 1 Day CAR-T Product Starting with Monocyte Depleted PBMCs:
A. Procurement of starting cells (day −1 or 0)
Autologous peripheral blood mononuclear cells were obtained from patients using leukapheresis (or a peripheral blood draw). Cell products were processed immediately or held overnight. (Cell product can also be cryopreserved if desired and processed at a later date).
B. Processing of apheresis sample (day 0)
The apheresis sample was washed by centrifugation to remove plasma and reduce the number of contaminating platelets. (If a peripheral blood draw was used instead of an apheresis sample then the PBMCs were isolated such as by ficoll based isolation method). Next, the apheresis sample was diluted in culture media such as TexMACS (Miltenyi Biotec) supplemented with CTS immune cell serum replacement (ThermoFisher Scientific), incubated in a tissue culture flask(s) or cell culture bags and placed in a culture incubator at 37° C. (±2° C.) to adhere monocytes at a concentration up to 5 million cells per ml. After adherence of monocytes to the flask(s) or bag(s) for at least 2 hours, non-adhered cells were transferred to new tissue culture flask(s) or bag(s). The volume was adjusted to 2 million cells per ml with culture media containing IL7 (10 ng/ml) and IL15 (5 ng/ml). (The culture media can also be prepared without the addition of any exogenous cytokine or with IL-7 alone, IL15 alone or other cytokines).
C. Activation of T cells (Day 0)
Cloudz® CD3/CD28 T cell activation reagent (R&D Systems/Biotechne) can be added to the flasks containing the monocyte depleted and processed apheresis product. (Other activation reagents including Soluble CD3 antibody, soluble CD3/CD28 antibody, surface bound CD3 antibody with or without CD28 soluble antibody, Immunocult® (Stemcell Technologies) or any other T cell activation reagent can be used).
D. Cell Transduction (Day 0)
Viral vector was added to the flasks or bags containing the monocyte depleted PBMCs immediately after addition of the T cell activation reagent using a multiplicity of infection (MOI) determined to lead to lead to a copy number per cell of less than 5. Cells were incubated in a 5% (±0.5%) CO₂ incubator at 37° C. (±2° C.) for 17-20 hr.

E. Cell harvest (Day 1)
GMP-grade 6×Release Buffer (R&D Systems/Biotechne) was added directly to the flasks or bags containing the activated/transduced cells if the cells were activated using the Cloudz® reagent. Next, the cells were washed and resuspended in freezing buffer such as Plasmalyte-A, 5% HSA and 5% DMSO in cryobag(s) or vial(s).
   Description of Method to Enable Vector Copy Number and Replication Competent Virus Release Testing with a Rapid Genetically Engineered T Cell Product:

As a key hurdle in the field of rapid T cell manufacturing workflows involving viral vectors is to perform required product release tests, the method described below was developed. Current required release test requirements for retroviral or lentiviral transduced cell therapy products include assessing replication competent lentivirus and vector copy number. Both of these assays have significant false positive results when there is even low level contamination with free plasmid when using the traditional qPCR based assays. Residual free plasmid from 293 cell transfections is present in viral vector. At early time points during manufacturing after viral transduction, there is residual plasmid remaining from the vector and false positive results are nearly impossible to eliminate using traditional testing methodologies. This false positive reactivity has been a major roadblock and limits the ability to rapidly manufacture products that can be used into patients.

As the DNA of interest for both viral integration and replication competent vector is viral DNA that is integrated, the manufacturing workflow included here is paired with a method to remove the free residual plasmid DNA so that the PCR reactions or other molecular testing can be performed for both vector copy number and replication competent lentivirus without this false positive reaction. In particular, free plasmid DNA is much smaller than genomic DNA. A centrifugation based method is used to deplete out the small DNA while maintaining the larger genomic DNA. Using this method there is no false positive reactivity from the plasmid DNA which overcomes a major hurdle for rapid CAR-T manufacturing while it is still feasible to measure DNA integrated into the genome.

In order to remove free plasmid DNA in CAR-T products that have been expanded for short periods (ex. 0-3 days), small sized DNA (ex. less than 10 kb) can be depleted while the genomic DNA can be maintained using centrifugation (ex. using solutions such as salt/polymer solutions to preferentially precipitate high molecular weight DNA). For example, the PacBio short read eliminator kit (PacBio; Menlo Park, California) can be used and enables this separation from a single centrifugation step (circulomics.com/store/Short-Read-Eliminator-Kit-p131401036). In addition to this commercial reagent, an alternate method is to utilize 4% PVP 360,000, 1.2 M KCL, 20 mM Tris-HCL ph 8 in the protocol described below instead of the commercial buffer SRE as it has been previously shown to effectively deplete small size fragments (ex. <10 kb from total DNA) (11). The purified DNA can then be used directly for the assay testing (ex. qPCR assays). While this kit was not designed for this particular indication, it functions well and provides a simple, cost effective and rapid approach.

One example of how this method can be employed is to initially isolate total DNA using any commercial total DNA isolation kit that can isolate human genomic DNA (ex. DNeasy Blood and Tissue Kit, Qiagen, Hilden, Germany). Next the following method or similar approach can be followed to remove low fragment DNA.

1. Using a wide bore pipette tip, Buffer SRE was added to the starting genomic DNA sample and mix thoroughly by pipetting. If the PacBio or other commercial reagents are not employed, 4% PVP 360,000, 1.2 M KCL, 20 mM Tris-HCL ph 8 can be added to the total DNA sample at a 1:1 volume ratio.
2. Centrifuged at 10,000×g for 30 minutes at room temperature.
3. Removed the supernatant with a pipette.
4. Added 70% ethanol to the tube.
5. Centrifuged tube at 10,000×g for around 2 minutes at room temperature.
6. Removed the supernatant and repeat the 70% ethanol wash
7. Added Buffer EB (or 10 mM Tris-HCL ph 8 if not using the PacBio kit) to the tube and incubate at 50° C. for 10-30 minutes and then resuspended the DNA pellet.

Using the low fragment removal workflow in combination with the rapid manufacturing workflow demonstrated that it was an effective process to enable release testing to be performed on genetically engineered T cell products at early timepoints of culture (ex. 17 hr to 3 days). For example, CAR-T product manufactured using the 17-20 hour workflow was harvested at 20 hr and a surrogate aliquot was harvested at 72 hours. Total DNA was isolated using the DNeasy Blood and Tissue Kit (Qiagen) and low fragment depleted DNA was prepared using the short read eliminator kit (PacBio). The low fragment depleted DNA as well as the total DNA samples were tested for replication competent lentivirus using a real-time PCR assay for VSV-G and vector integration using a real-time PCR assay for GAG and the housekeeping gene PTBP2. The primers and fluorescent probes used for the PCR reactions can be found in Table 6.

Table 6. PCR primers and Probes used for the GAG, VSV-G and PTPBP2 qPCR assays.

GAG Primers
5′-GGA GCT AGA ACG ATT CGC AGT TA- 3′
5′ GGT TGT AGC TGT CCC AGT ATT TGT C- 3′
GAG Probe
5′-/56-FAM/ACA GCC TTC TGA TGT TTC TAA CAG GCC AGG
3′BHQ-1
PTPB2 Primers
5′-TCT CCA TTC CCT ATG TTC ATG C-3′
5′-GTT CCC GCA GAA TGG TGA GGT G-3′
PTPB2 Probe
5′-/5HEX/ATG TTC CTC /ZEN/GGA CCA ACT TG 3iabfq 3′
VSVG Primers
5′-AGG GAA CTG TGG GAT GAC TG
5′-GAA CAC CTG AGC CTT TGA GC
VSVG Probe
5′-6FAM-GAACACCTGAGCCTTTGAGC MGBNFQ-3′

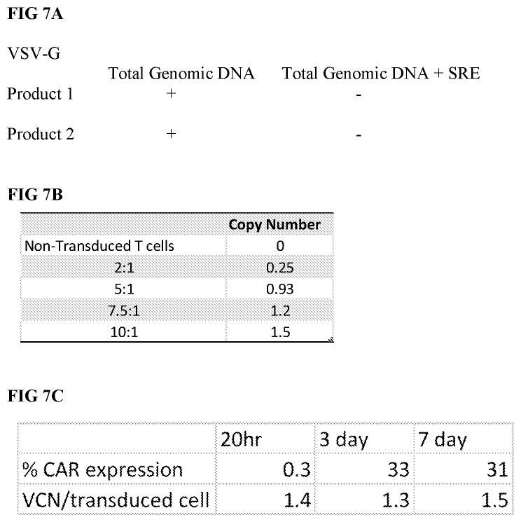

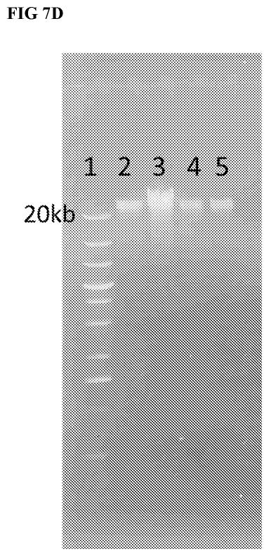

The PCR products were quantitated using a standard probe that consisted of a linearized plasmid engineered to express single copies of GAG, VSV-G and PTBP2. As seen in FIG. 7A, the qPCR reactions performed with the low fragment depleted DNA did not detect VSV-G and therefore were negative for replication competent lentivirus as expected on two different products manufactured for 20 hours. In contrast, when total genomic DNA was utilized without low fragment depletion, both samples gave false positive qPCR results for VSV-G likely due to the low fragment free plasmid contamination. To assess if alternate approaches such as cell washing or culture for 3 days (as opposed to 20 hours) could overcome the false positive reactivity with the replication competent lentivirus testing, we performed further assays. In particular, we harvested lentivirally transduced CD19 CAR-T products at 20 hours and 72 hours and performed extensive washing of the cells prior to genomic DNA isolation. In particular, the cells (5×10⁶ cells) were washed in a volume of 50 ml PBS 15 times for the 72 hour samples and 5 times for the 20 hour samples. In all cases (20 and 72 hour samples for both products), there was false positive reactivity using the VSV-G qPCR assay. Therefore, increased culture duration to 3 days and extensive cell washing is not sufficient to reduce this false positive reactivity.

Of note for replication competent lentivirus testing, there has never been a positive test reported to the FDA in the past 10 years for clinical products reinforcing the positive reactivity consistently observed with the rapid product manufacturing at 17-20 hr and 72 hours is due to false positive reactivity (fda.gov/media/113790/download). In addition, when culture durations are extended to 8 days, the VSV-G qPCR assays become negative suggesting that the free plasmid DNA is lost by that timepoint and that true replication competent virus was not present.

Similar to replication competent lentivirus testing, another important release test for virally transduced cell therapy products is vector integration which is often measured by qPCR against the GAG gene for which free plasmid contamination is also present in viral vector and can give false positive reactivity. Initially, GAG/PTPB2 qPCR was performed to determine the vector copy number on CD19 CAR lentivirally transduced T cells that were maintained in culture for 8 days in order to assess the multiplicity of infection (MOI) of the vector and to assess a baseline for full vector integration. As see in FIG. 7B, an MOI of 10:1 led to a peak vector copy number per transduced T cell (i.e., CD19 CAR expressing) of 1.5. Next the vector copy number per transduced cell was determined using the rapid manufacturing workflow when employing an MOI of the CD19 CAR vector of 10:1 except the cells were harvested at 20 hours, 3 days and 7 days. In addition, the SRE kit (PacBio) was utilized to remove low fragment DNA. As seen in FIG. 7C, the vector integration results were similar across all time points. Of note CD19 CAR surface expression is very low at 20 hours, though it is fully expressed around 72 hours.

Finally, gel electrophoresis of total DNA samples prepared from two 20 hour rapid manufactured T cell products before and after low fragment removal demonstrate that there is maintenance of the genomic DNA as expected and therefore only the undesirable low molecular weight fragments are depleted (FIG. 7D). In addition, quantification of the DNA concentration using the Nanodrop® spectrophotometer (ThermoFisher Scientific) did not reveal any measureable change in DNA concentration before and after low fragment removal demonstrating that only minute amounts of DNA are removed.

As will be appreciated, the presently described CAR-T cell manufacturing workflow provides significant improvements over prior art methodologies. In particular, without intending any limitation:

1. Primarily performs manufacturing using unfractionated cells from an apheresis product or apheresis cells that underwent monocyte depletion by adherence to a solid surface. In addition, peripheral blood mononuclear cells (PBMCs) or monocyte depleted PBMCs directly isolated from peripheral blood can be utilized. Therefore, it does not require purified T cells that was assumed to be important for a rapid CAR-T workflow involving viral transduction. As far as we are aware, this is the first 1 day or less protocol employing PBMCs and not isolated T cells as a starting source for rapid genetically modified T cell manufacturing using viral transduction.
2. Cell activation is performed using different methods. Magnetic beads take days to fall off cells and therefore methodologies employing such beads either delays the day when products can be harvested and/or results in a significantly reduced product yield due to loss of bead bound cells. In addition, unexpectedly we observed enhanced transduction efficiency using specific non-magnetic bead based T cell activation reagents. The presently described activation approaches provide improved transduction efficiency coupled with the avoidance of increased costs and increased manufacturing complexity involved with utilizing magnetic beads as required in prior art methods.
3. The presently described manufacturing process is the first workflow rapid CAR-T manufacturing workflow described that can be performed efficiently in the absence of exogenous cytokines. Cytokines during manufacturing add significantly increased costs and can also lead to changes in the T cells. As a major benefit of the rapid manufacturing workflow is to maintain the starting population of T cells (ex. naïve T cells) as close to possible as the original cells collected from the patient, the avoidance of exogenous cytokines is viewed as a major benefit.
4. The described use of a fully closed system to manufacture the T cell product using bags allows providing a convenient kit based product in which the virus/media/activation reagent etc. would be provided in a bag and then mixed with the apheresis product. The product could be manufactured using this method outside of a clean room to facilitate widespread use and manufacturing of the product which can reduce costs and increase accessibility.
5. The presently described methods overcome a key hurdle in the field of performing required product release tests. Current required release test requirements for retroviral or lentiviral transduced cell therapy products include assessing replication competent lentivirus and vector copy number. Both of these assays have significant false positive results when there is even low level contamination with free plasmid when using the traditional qPCR based assays. Residual free plasmid from viral producer cells such as 293 cells is present in viral vector. At early time points during manufacturing after viral transduction, there is residual plasmid remaining from the vector and false positive results are nearly impossible to eliminate using traditional testing methodologies. This false positive reactivity has been a major roadblock for other groups and limits the ability to rapidly manufacture products that can be used into patients. As the DNA of interest for both viral integration and replication competent vector is viral DNA that is integrated, the manufacturing workflow included here is paired with a method to remove the free residual plasmid DNA so that the qPCR reactions can be performed for both vector copy number and replication competent lentivirus without this false positive reaction. In particular, free plasmid DNA is much smaller than genomic DNA. A centrifugation based method is used to deplete out the small DNA while maintaining the larger genomic DNA. Using this method there is no false positive reactivity from the plasmid DNA which overcomes a major hurdle for rapid CAR-T manufacturing while it is still feasible to measure DNA integrated into the genome.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9):1726-1732).

Although any methods, devices, and materials similar or equivalent to those described herein can be used in the practice or testing of the presently-disclosed subject matter, representative methods, devices, and materials are described herein.

In certain instances, nucleotides and polypeptides disclosed herein are included in publicly-available databases, such as GENBANK© and SWISSPROT. Information including sequences and other information related to such nucleotides and polypeptides included in such publicly-available databases are expressly incorporated by reference. Unless otherwise indicated or apparent the references to such publicly-available databases are references to the most recent version of the database as of the filing date of this Application.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

Wherever any of the phrases “for example,” “such as,” “including” and the like are used herein, the phrase “and without limitation” is understood to follow unless explicitly stated otherwise. Similarly “an example,” “exemplary” and the like are understood to be non-limiting. The term “substantially” allows for deviations from the descriptor that do not negatively impact the intended purpose. Descriptive terms are understood to be modified by the term “substantially” even if the word “substantially” is not explicitly recited. Therefore, for example, the phrase “wherein the lever extends vertically” means “wherein the lever extends substantially vertically” so long as a precise vertical arrangement is not necessary for the lever to perform its function.

The terms “comprising” and “including” and “having” and “involving” (and similarly “comprises”, “includes,” “has,” and “involves”) and the like are used interchangeably and have the same meaning. Specifically, each of the terms is defined consistent with the common United States patent law definition of “comprising” and is therefore interpreted to be an open term meaning “at least the following,” and is also interpreted not to exclude additional features, limitations, aspects, etc. Thus, for example, “a process involving steps a, b, and c” means that the process includes at least steps a, b and c. Wherever the terms “a” or “an” are used, “one or more” is understood, unless such interpretation is nonsensical in context. The terms “comprise”, “have”, “include” and “contain” (and their variants) are open-ended linking verbs and allow the addition of other elements when used in a claim.

The use of the word “a” or “an” when used in conjunction with the term “comprising” in the claims or the specification means one or more than one, unless the context dictates otherwise.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, in some embodiments ±0.1%, in some embodiments ±0.01%, and in some embodiments ±0.001% from the specified amount, as such variations are appropriate to perform the disclosed method. The use of the term “or” in the claims is used to mean “and/or” unless explicitly indicated to refer to alternatives only or if the alternatives are mutually exclusive.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

It will be understood that various details of the presently disclosed subject matter can be changed without departing from the scope of the subject matter disclosed herein. Furthermore, the foregoing description is for the purpose of illustration only, and not for the purpose of limitation. Obvious modifications and variations are possible in light of the above teachings. All such modifications and variations are within the scope of the appended claims when interpreted in accordance with the breadth to which they are fairly, legally and equitably entitled.

### REFERENCES

1. Rodriguez J, Caballero M D, Gutierrez A, Solano C, Arranz R, Lahuerta J J, Sierra J, Gandarillas M, Perez-Simon J A, Zuazu J, Lopez-Guillermo A, Sureda A, Carreras E, Garcia-Larana J, Marin J, Garcia J C, Fernandez De Sevilla A, Rifon J, Varela R, Jarque I, Albo C, Leon A, SanMiguel J, Conde E. Autologous stem-cell transplantation in diffuse large B-cell non-Hodgkin's lymphoma not achieving complete response after induction chemotherapy: the GEL/TAMO experience. Ann Oncol. 2004; 15(10):1504-9. Epub 2004/09/16. doi: 10.1093/annonc/mdh391. PubMed PMID: 15367411.
2. Vose J M, Zhang M J, Rowlings P A, Lazarus H M, Bolwell B J, Freytes C O, Pavlovsky S, Keating A, Yanes B, van Besien K, Armitage J O, Horowitz M M, Autologous B, Marrow Transplant Registry Lymphoma Working C. Autologous transplantation for diffuse aggressive non-Hodgkin's lymphoma in patients never achieving remission: a report from the Autologous Blood and Marrow Transplant Registry. J Clin Oncol. 2001; 19(2):406-13. Epub 2001/02/24. doi: 10.1200/JCO.2001.19.2.406. PubMed PMID: 11208832.
3. Crump M, Neelapu S S, Farooq U, Van Den Neste E, Kuruvilla J, Westin J, Link B K, Hay A, Cerhan J R, Zhu L, Boussetta S, Feng L, Maurer M J, Navale L, Wiezorek J, Go W Y, Gisselbrecht C. Outcomes in refractory diffuse large B-cell lymphoma: results from the international SCHOLAR-1 study. Blood. 2017; 130(16):1800-8. Epub 2017/08/05. doi: 10.1182/blood-2017-03-769620. PubMed PMID: 28774879; PMCID: PMC5649550.
4. Westin J R, Kersten M J, Salles G, Abramson J S, Schuster S J, Locke F L, Andreadis C. Efficacy and Safety of CD19-Directed CAR-T Cell Therapies in Patients With Relapsed/Refractory Aggressive B-Cell Lymphomas: Observations From the JULIET, ZUMA-1, and TRANSCEND Trials. American journal of hematology. 2021. Epub 2021/07/27. doi: 10.1002/ajh.26301. PubMed PMID: 34310745.
5. Harrison R P, Zylberberg E, Ellison S, Levine B L. Chimeric antigen receptor-T cell therapy manufacturing: modelling the effect of offshore production on aggregate cost of goods. Cytotherapy. 2019; 21(2):224-33. Epub 2019/02/17. doi: 10.1016/j.jcyt.2019.01.003. PubMed PMID: 30770285.
6. Neelapu S S, Locke F L, Bartlett N L, Lekakis L J, Miklos D B, Jacobson C A, Braunschweig I, Oluwole 00, Siddiqi T, Lin Y, Timmerman J M, Stiff P J, Friedberg J W, Flinn I W, Goy A, Hill B T, Smith M R, Deol A, Farooq U, McSweeney P, Munoz J, Avivi I, Castro J E, Westin J R, Chavez J C, Ghobadi A, Komanduri K V, Levy R, Jacobsen E D, Witzig T E, Reagan P, Bot A, Rossi J, Navale L, Jiang Y, Aycock J, Elias M, Chang D, Wiezorek J, Go W Y. Axicabtagene Ciloleucel CAR T-Cell Therapy in Refractory Large B-Cell Lymphoma. N Engl J Med. 2017; 377(26):2531-44. Epub 2017/12/12. doi: 10.1056/NEJMoa1707447. PubMed PMID: 29226797; PMCID: PMC5882485.
7. Kersten M J, Spanjaart A M, Thieblemont C. CD19-directed CAR T-cell therapy in B-cell NHL. Curr Opin Oncol. 2020; 32(5):408-17. Epub 2020/08/03. doi: 10.1097/CCO.0000000000000668. PubMed PMID: 32740094.
8. Zhang H, Zhao P, Huang H. Engineering better chimeric antigen receptor T cells. Exp Hematol Oncol. 2020; 9(1):34. Epub 2020/12/10. doi: 10.1186/s40164-020-00190-2. PubMed PMID: 33292660; PMCID: PMC7709221.
9. Li Y, Kurlander R J. Comparison of anti-CD3 and anti-CD28-coated beads with soluble anti-CD3 for expanding human T cells: differing impact on CD8 T cell phenotype and responsiveness to restimulation. Journal of translational medicine. 2010; 8:104. Epub 2010/10/28. doi: 10.1186/1479-5876-8-104. PubMed PMID: 20977748; PMCID: PMC2987859.
10. Schuster S J, Bishop M R, Tam C S, Waller E K, Borchmann P, McGuirk J P, Jager U, Jaglowski S, Andreadis C, Westin J R, Fleury I, Bachanova V, Foley S R, Ho P J, Mielke S, Magenau J M, Holte H, Pantano S, Pacaud L B, Awasthi R, Chu J, Anak O, Salles G, Maziarz R T, Investigators J. Tisagenlecleucel in Adult Relapsed or Refractory Diffuse Large B-Cell Lymphoma. The New England journal of medicine. 2019; 380(1):45-56. Epub 2018/12/07. doi: 10.1056/NEJMoa1804980. PubMed PMID: 30501490.
11. Jones A, Torkel C, Stanley D, Nasim J, Borevitz J, Schwessinger B. High-molecular weight DNA extraction, clean-up and size selection for long-read sequencing. PloS one. 2021; 16(7):e0253830. Epub 2021/07/16. doi: 10.1371/journal.pone.0253830. PubMed PMID: 34264958; PMCID: PMC8282028.

1. A method for rapid manufacture of a genetically modified T-cell population, comprising:
obtaining a mixed mononuclear cell population; and
substantially simultaneously:
exposing a T-cell population comprised in the mixed mononuclear cell population to a T-cell activation agent; and
exposing the mixed mononuclear cell population to a viral vector adapted to transduce at least the T-cell population comprised in the mixed mononuclear cell population with a foreign nucleotide.

2. The method of claim 1, including harvesting the mixed mononuclear cell population comprising at least a genetically modified T-cell population at up to 24 hours from the steps of simultaneously activating and exposing to at least one viral vector.

3. The method of claim 1, wherein the activating is performed by exposing the mixed mononuclear cells to the T-cell activation agent selected from one or more of the group of cytokines consisting of IL-2, IL-7, IL-15, IL-12, IL-18, and IL21, and/or to activators for one or more of CD3, CD28, OX40, CD2, CD27, ICAM-1, LFA-1 (CD11a/CD18), ICOS (CD278), and 4-1BB (CD137).

4. The method of claim 3, wherein the activating is performed by exposing the mixed mononuclear cells to the T-cell activation agent selected from one or more of the group consisting of IL-7 and IL-15.

5. The method of claim 1, including a step of at least partial depletion by adherence of a monocyte population comprised in the mononuclear cell population prior to the steps of activating and exposing to the viral vector.

6. The method of claim 5, wherein the steps of activating and exposing to the viral vector are performed in the absence of any exogenous cytokine.

7. The method of claim 6, wherein the step of activating is performed by exposing the mixed mononuclear cell population to one or more of a CD3 activator, a CD28 activator, soluble or surface-bound CD3 antibody, or soluble or surface-bound CD28 antibody.

8. The method of claim 7, wherein the step of activating is performed by exposing the mixed mononuclear cell population to one or both of CD3 activator or soluble or surface-bound CD3 antibody.

9. The method of claim 1, wherein the steps of activating and exposing to the viral vector are performed without a prior T-cell isolation or pre-activation step.

10. The method of claim 1, further including cryopreserving the mixed mononuclear cell population comprising the genetically modified T-cell population.

11. The method of claim 1, including obtaining the mixed mononuclear cell population by apheresis or a peripheral blood draw.

12. The method of claim 1, including selecting the viral transduction vector from the group consisting of a lentivirus, a retrovirus, and an adenovirus.

13. The method of claim 1, further including a step of differential centrifugation following the step of substantially simultaneously activating and exposing to the viral transduction vector to remove a plasmid DNA from genomic DNA by DNA size selection.

14. A genetically modified T-cell, produced by the method of claim 1.

15. A closed system kit for performing the method for rapid manufacture of a genetically modified T-cell population according to claim 1, comprising:
a first sterile vessel adapted for receiving a mixed mononuclear cell population;
a second sterile vessel adapted for receiving a mixed mononuclear cell population depleted of monocytes;
a bead-free T-cell activation agent;
a viral vector adapted to transduce a T-cell population with a foreign nucleotide;
a suitable culture media; and
a suitable cell washing solution;
wherein at least the first vessel is fabricated of a material suitable for depletion of monocytes from the mixed mononuclear cell population;
further wherein the first and second vessels are adapted for sterile introduction of the bead-free T-cell activation agent, the viral transduction vector, the culture media, and the cell washing solution.

16. The closed system kit of claim 15, wherein the bead-free T-cell activation agent is selected from one or more of the group consisting of a CD3 activator, a CD28 activator, a OX40 activator, a CD2 activator, a CD27 activator, a ICAM-1 activator, a LFA-1 (CD11a/CD18) activator, a ICOS (CD278) activator, a 4-1BB (CD137) activator, and CD3 antibody.

17. The closed system kit of claim 16, further including one or more cytokines selected from the group consisting of IL-2, IL-7, and IL-15.

18. The closed system kit of claim 16, wherein the bead-free T-cell activation agent is selected from one or more of the group consisting of one or more of soluble or surface-bound CD3 antibody and soluble or surface-bound CD28 antibody.

19. The closed system kit of claim 18, wherein the bead-free T-cell activation agent is selected from one or more of the group consisting of one or more of soluble or surface-bound CD3 antibody.

20. The closed system kit of claim 15, wherein the viral vector is selected from the group consisting of a lentivirus, a retrovirus, and an adenovirus.

21. The closed system kit of claim 15, wherein one or both of the first and the second vessel are selected from the group consisting of a cell culture bag and a cell culture flask.
